(19) United States
(12) Patent Application Publication
(10) Pub. No.: US 2022/0023460 A1
(43) Pub. Date: Jan. 27, 2022

(54) WATERPROOF DISINFECTION LAMP

(71) Applicant: MOU-LI LIN, TAINAN CITY (TW)

(72) Inventor: MOU-LI LIN, TAINAN CITY (TW)

(21) Appl. No.: 17/032,589

(22) Filed: Sep. 25, 2020

(51) Int. Cl. A61L 2/10; A61L 2/26

(52) U.S. Cl. CPC A61L 2/10; A61L 2202/11; A61L 2/26

(57) ABSTRACT

A waterproof disinfection lamp includes a housing, a light-emitting member, a power supply module, a cover, and a gasket. One side of the housing is formed with a recess. The light-emitting member is disposed on the housing and controlled to generate an ultraviolet light. The power supply module is disposed in the recess. The cover is detachably coupled to the recess. When the cover is coupled to the recess, the gasket is disposed between the recess and the cover to form a seal between the recess and the cover.

### FIELD OF THE INVENTION

The present invention relates to a disinfection lamp, and more particularly to a waterproof disinfection lamp.

### BACKGROUND OF THE INVENTION

There is a laundry ball device with a disinfection and sterilization function on the market, referring to Chinese Patent Publication No. CN102021782. The laundry ball device includes a body structure, a light-emitting diode module, and a power module. The light-emitting diode module emits a light source with a certain wavelength to provide a disinfection and sterilization function. The, power supply module is disposed on the body structure and electrically connected to the light-emitting diode module. The power module supplies the power required by the light-emitting diode module.

When a washing machine is used for washing clothes, the laundry ball device can be thrown into the washing machine together with the clothes for washing. The light-emitting diode module emits ultraviolet rays, which has a good effect on disinfection and sterilization. Therefore, the clothes are disinfected and sterilized during the washing process. However, there are circuits, such as the light-emitting diode module and the power module, inside the laundry ball device. During the washing process, in order to prevent water from entering the, inside, of the laundry ball device, the body structure is designed to be closed and cannot be opened. The power module is charged by wireless charging technology. However, this charging manner increases the manufacturing cost of the laundry ball device.

### SUMMARY OF THE INVENTION

Therefore, in order to solve the problem of high production cost caused by the use of wireless charging, the primary object of the present invention is to provide a waterproof disinfection lamp. The waterproof disinfection lamp comprises a housing, a light-emitting member, a power supply module, a cover, and a gasket.

One side of the housing is formed with a recess. The recess includes a bottom wall. The light-emitting member is disposed on the housing and controlled to generate an ultraviolet light. The power supply module includes a charging interface. The power supply module is disposed inside the housing and electrically connected to the light-emitting member. The charging interface is disposed in the recess. The cover includes a coupling wall. The cover is detachably coupled to the recess. When the cover is coupled to the recess, the gasket is disposed between the bottom wall and the coupling wall to form a seal between the bottom wall and the coupling wall.

Preferably, the recess is formed with an internal thread, the cover is formed with an external thread, and the cover is coupled to the recess with the external thread to engage with the internal thread.

Preferably, the bottom wall is formed with an annular groove. The charging interface is disposed on the bottom wall and surrounded by the annular groove. When the cover is coupled to the recess, the gasket is positioned in the annular groove.

Preferably, the recess defines an interior space. The cover further includes a rib. When the cover is coupled to the recess, the rib is exposed in the interior space. The rib is formed with a rope hole.

Preferably, the power supply module further includes a rechargeable battery and a charging indicator. The rechargeable battery is disposed inside the housing. The charging indicator is disposed in the recess. The cover has light permeability. When the power of the rechargeable battery is lower than a required charging value, the charging indicator emits a first light warning. When the charging interface is electrically connected to an external power source for charging, the rechargeable battery is in a charging state, and the charging indicator emits a second light warning. When the rechargeable battery is charged and the power reaches a full charge value, the charging indicator emits a third light warning.

Preferably, the waterproof disinfection lamp further comprises a switch module. The switch module is disposed inside the housing and is electrically connected between the power supply module and the light-emitting member. The switch module includes a switch and a function indicator. The switch is disposed on the housing and has light permeability. The function indicator is covered by the switch. When the switch is on, the function indicator emits a light warning within a delay time. After the function indicator goes out, the light-emitting member starts to emit the ultraviolet light. The switch is off after a predetermined time, so that the light-emitting member goes out after the predetermined time. When the switch is off, the function indicator and the light-emitting member go out.

Preferably, the switch is a mechanical self-locking switch.

According to the above technical features, the following effects can be achieved:

1. The gasket is arranged between the bottom wall and the coupling wall to form a seal between the bottom wall and the coupling wall. When the waterproof disinfection lamp is thrown into water, the water cannot penetrate into the charging a interface and enter the inside of the housing.

2. The gasket is arranged in the annular groove. When the cover is coupled to the recess, the gasket is positioned in the annular groove.

3. The cover is coupled to the recess with the external thread to engage with the internal thread. This is convenient for assembly.

### BRIEF DESCRIPTION OF THE DRAWINGS

FIG. 1 is an exploded view of a waterproof disinfection lamp according to an embodiment of the present invention;

FIG. 2 is a perspective view of the waterproof disinfection lamp according to the embodiment of the present invention;

FIG. 3 is another perspective view of the waterproof disinfection lamp according to the embodiment of the present invention;

FIG. 4 is a block diagram illustrating a plurality of light-emitting members, a power supply module and a switch module according to the embodiment of the present invention;

FIG. 5 is a schematic view illustrating that a plurality of waterproof disinfection lamps are placed in a washing machine;

FIG. 6 is a schematic view illustrating that the waterproof disinfection lamps is hung in a closet; and

FIG. 7 is a schematic view illustrating that the waterproof disinfection lamps is hung in a car.

### DETAILED DESCRIPTION OF THE PREFERRED EMBODIMENTS

Embodiments of the present invention will now be described, by way of example only, with reference to the accompanying drawings.

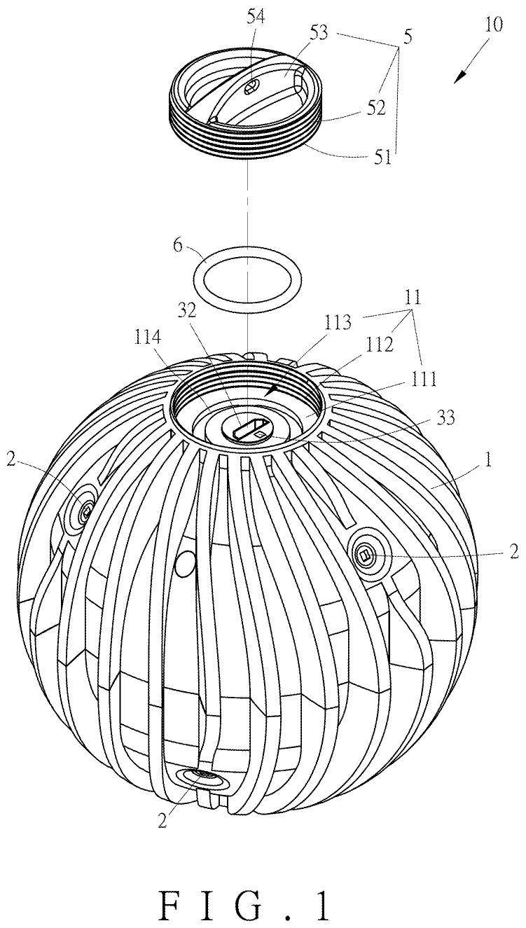

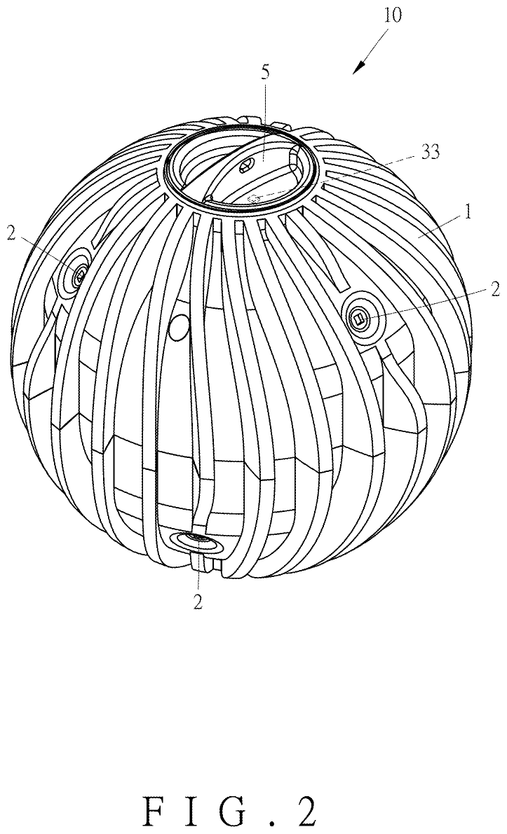

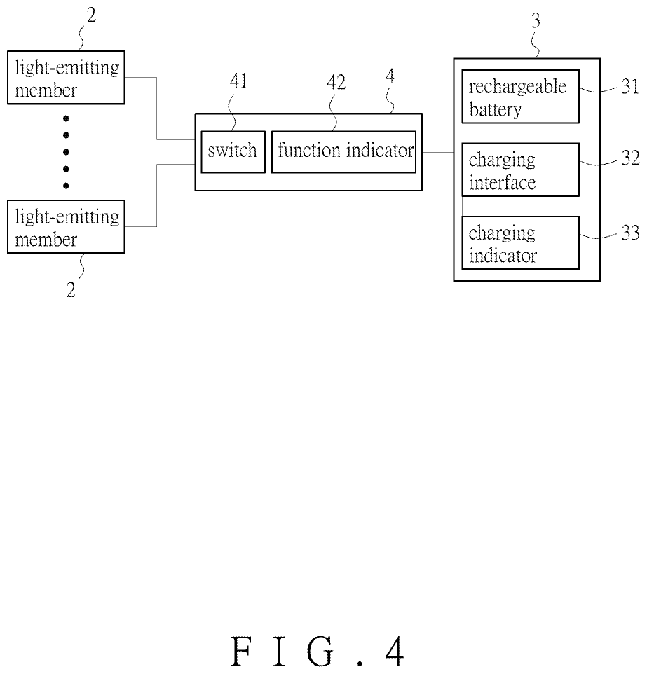

As shown in FIG. 1, FIG. 2 and FIG. 4, a waterproof disinfection lamp 10 according to an embodiment of the present invention comprises a housing I, a plurality of light-emitting members 2, a power supply module 3, a switch module 4, a cover 5, and a gasket 6.

The housing 1 has a spherical shape. One side of the housing 1 is formed with a recess 11. The recess 11 includes a bottom wall 111 and a first circumferential wall 112, and defines an interior space 113. The bottom wall 111 is formed with an annular groove 114. The first circumferential wall 112 is formed with internal threads.

The light-emitting members 2 are spaced and arranged on the housing 1. Each light-emitting member 2 is controlled to generate an ultraviolet light. In this embodiment, each light-emitting member 2 is a light-emitting diode. Before being mounted to the housing 1, the light-emitting diode has been waterproofed. The ultraviolet light generated by each light-emitting member 2 can achieve a disinfection effect.

The power supply module 3 includes a rechargeable battery 31, a charging interface 32. and a charging indicator 33. The rechargeable battery 31 is disposed inside the housing I. The charging interface 32 and the charging indicator 33 are disposed on the bottom wall 111 and surrounded by the annular groove 114. In this embodiment, the charging interface 32 is a universal serial bus (USB) connector, but it is not limited to this.

The switch module 4 is disposed inside the housing 1, and is electrically connected between the power supply module 3 and the light-emitting members 2. The switch module 4 includes a switch 41 and a function indicator 42. The switch 41 is disposed on the housing 1 and has light permeability. In this embodiment, the switch 41 is a mechanical self-locking switch. The outer layer of the switch 41 is a silicon gel layer, so that the switch 41 has light permeability. The function indicator 42 is covered by the switch 41, but the light of the function indicator 42 can penetrate the switch 41.

The cover 5 includes a coupling wall 51, a second circumferential wall 52, and a rib 53. The coupling wall 51 and the rib 53 are located on opposite sides of the cover 5. The second circumferential wall 52 is formed with external threads. The rib 53 has a rope hole 54. The cover 5 has light permeability.

The gasket 6 is disposed in the, annular groove 114.

When assembling, the cover 5 is detachably coupled to the recess 11 with the external threads to engage with the internal threads, and is accommodated in the interior space 113. The gasket 6 is disposed between the bottom wall 111 and the coupling wall 51 to form a seal between the bottom wall 111 and the coupling wall 51. Therefore, when the waterproof disinfection lamp 10 is thrown into the washing machine and washed together with the clothes, the gasket 6 is tightly sealed so that water cannot penetrate into the charging interface 32 to affect the power supply module 3 and the switch module 4 inside the housing 1.

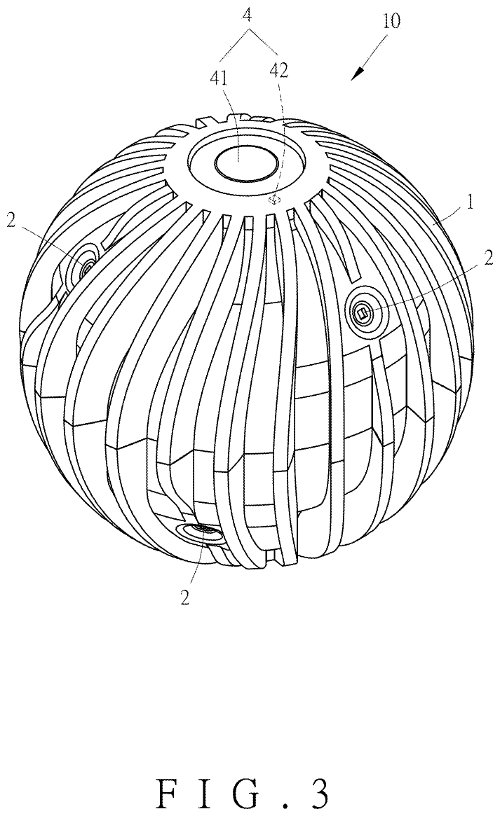

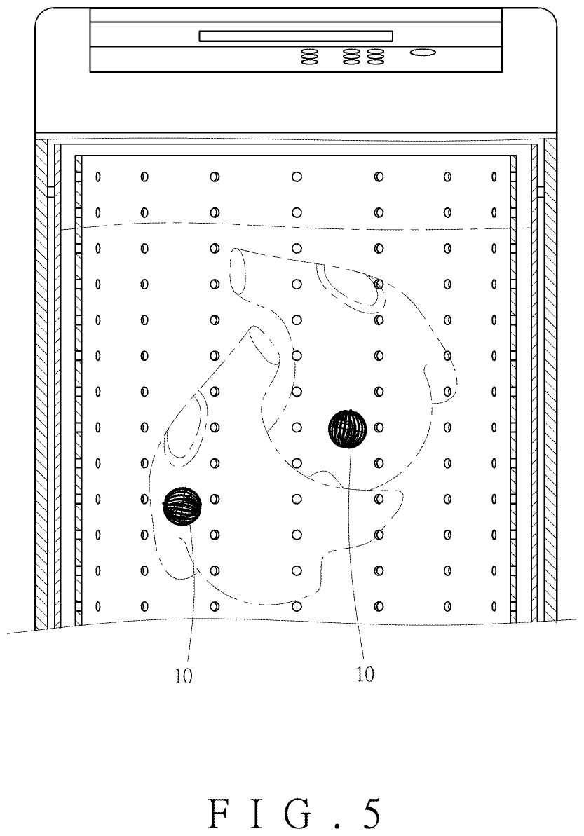

Referring to FIG. 1, FIG. 3 and FIG. 5, when the waterproof disinfection lamp 10 is in use, the switch 41 is first pressed. Since the switch 41 is a mechanical self-locking switch, it needs to be pressed with a certain amount of force, and it will be off automatically after a predetermined time to avoid accidental touch or being played by children. When the switch 41 is on, the function indicator 42 will emit a light warning within a delay time. At this time, the waterproof disinfection lamp 10 can be thrown into the washing machine. After the function indicator 42 goes out, the light-emitting members 2 start to emit ultraviolet light. The light-emitting members 2 generate ultraviolet light to eliminate bacteria and viruses on the clothes that are washed together. The switch 41 is off after a predetermined time, so that the light-emitting members 2 go out after the predetermined time. When the switch 41 is off, the function indicator 42 and the light-emitting members 2 go out. In this embodiment, the delay time is 10 seconds. The light warning is a flashing blue light. The light of the function indicator 42 can penetrate the switch 41 and is exposed outside the housing 1 to remind the user that the light-emitting members 2 are about to emit ultraviolet light, so as to prevent the user from being injured by the ultraviolet light without noticing that the light-emitting members 2 have been turned on. The predetermined time is one hour. The light-emitting members 2 generate the ultraviolet light for one hour and then automatically go out. When the user wants to use the waterproof disinfection lamp 10 again, the user presses the switch 41 to make the switch 41 bounce back to its original position. The user continues to press the switch 41 to keep the switch 41 on. The switch 41 is off after the predetermined time, so as to prevent the user from forgetting to turn off the switch 41 after use, which will cause a waste of power in the rechargeable battery 31 (referring to FIG. 4).

Referring to FIG. 1, FIG. 2, and FIG. 4, since the cover 5 has light permeability, the light of the charging indicator 33 can penetrate the cover 5 and be exposed outside the housing 1. When the power of the rechargeable battery 3 is lower than a required charging value, the charging indicator 33 emits a first light warning, and the first light warning is, for example, a flashing red light. When the user sees the flashing red light, he/she knows that the waterproof disinfection lamp 10 needs to be charged. When charging, the user only needs to unscrew the cover 5 and take it out from the interior space 113, and the charging interface 32 is exposed. This is quite convenient. Then, the charging interface 32 is electrically connected to an external power source for charging. When the rechargeable battery is in a charging state, the charging indicator 33 emits a second light warning, and the second light warning is, for example, a steady red light. The user knows that the rechargeable battery is not fully charged when he/she sees the steady red light. When the rechargeable battery 31 is charged and the power reaches a full charge value, the charging indicator 33 emits a third light warning. The third light warning is, for example, a steady blue light. The user knows that the rechargeable battery 31 has been charged when he/she sees the steady blue light, and the power source can be removed, and the cover 5 is coupled to the recess 11.

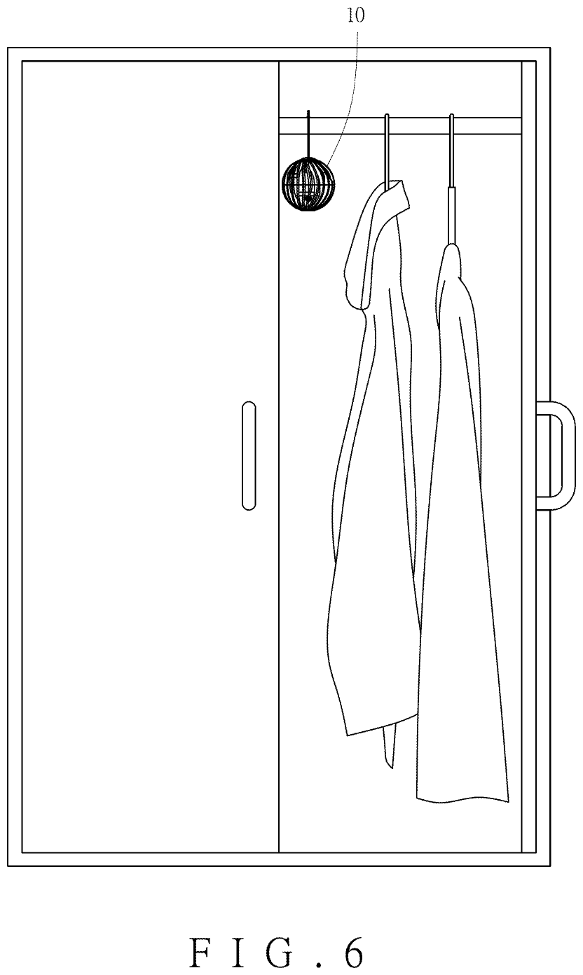

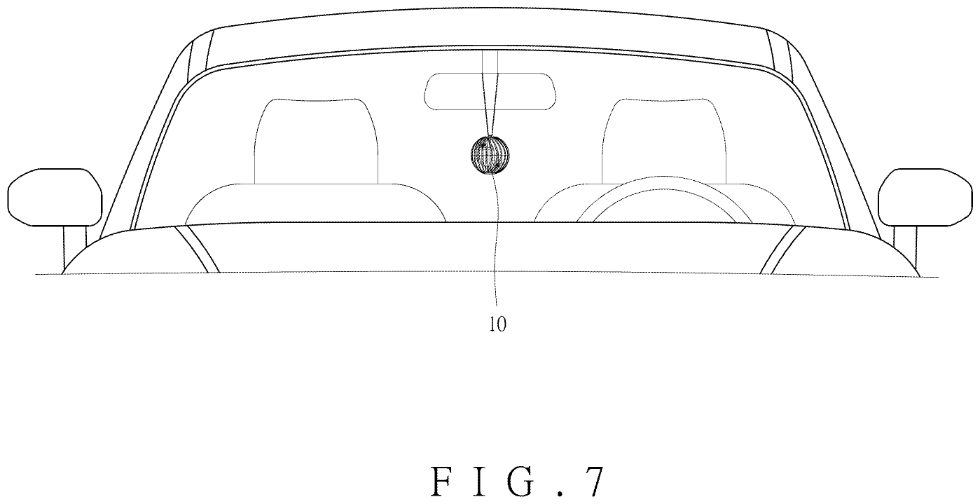

Referring to FIG. 1, FIG. 6 and FIG. 7, the waterproof disinfection lamp 10 emits the ultraviolet light through the light-emitting members 2 and has a good disinfection effect. In addition to being used in a washing machine, the waterproof disinfection lamp 10 can be hung in a closet or a car through the rope hole 54 so that the space in the closet or the car can also be disinfected and sterilized. The waterproof disinfection lamp 10 can be placed or hung in any space to be disinfected.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A waterproof disinfection lamp, comprising:
a housing, one side of the housing being formed with a recess, the recess including a bottom wall;
a light-emitting member, disposed on the housing and controlled to generate an ultraviolet light;
a power supply module, including a charging interface, the power supply module being disposed inside the housing and electrically connected to the light-emitting member, the charging interface being disposed in the recess;
a cover, including a coupling wall, the cover being detachably coupled to the recess;
a gasket, wherein when the cover is coupled to the recess, the gasket is disposed between the bottom all and the coupling wall to form a seal between the bottom wall and the coupling wall.

2. The waterproof disinfection lamp as claimed in claim 1, wherein the recess is formed with an internal thread, the cover is formed with an external thread, and the cover is coupled to the recess with the external thread to engage with the internal thread.

3. The waterproof disinfection lamp as claimed in claim 1, wherein the bottom wall is formed with an annular groove, the charging interface is disposed on the bottom wall and surrounded by the annular groove, when the cover is coupled to the recess, the gasket is positioned in the annular groove.

4. The waterproof disinfection lamp as claimed in claim 1, wherein the recess defines an interior space, the cover further includes a rib, when the cover is coupled to the recess, the rib is exposed in the interior space, and the rib is formed with a rope hole.

5. The waterproof disinfection lamp as claimed in claim 1, wherein the power supply module further includes a rechargeable battery and a charging indicator, the rechargeable battery is disposed inside the housing, the charging indicator is disposed in the recess, the cover has light permeability, when the power of the rechargeable battery is lower than a required charging value, the charging indicator emits a first light warning; when the charging interface is electrically connected to an external power source for charging, the rechargeable battery is in a charging state, and the charging indicator emits a second light warning; when the rechargeable battery is charged and the power reaches a full charge value, the charging indicator emits a third light warning.

6. The waterproof disinfection lamp as claimed in claim 1, further comprising a switch module, wherein the switch module is disposed inside the housing and is electrically connected between the power supply module and the light-emitting member, the switch module includes a switch and a function indicator, the switch is disposed on the housing and has light permeability, the function indicator is covered by the switch, when the switch is on, the function indicator emits a light warning within a delay time, after the function indicator goes out, the light-emitting member starts to emit the ultraviolet light, the switch is off after a predetermined time, so that the light-emitting member goes out after the predetermined time, when the switch is off, the function indicator and the light-emitting member go out.

7. The waterproof disinfection lamp as claimed in claim 6, wherein the switch is a mechanical self-locking switch.
